(19) United States
(12) Patent Application Publication
(10) Pub. No.: US 2023/0197279 A1
(43) Pub. Date: Jun. 22, 2023

(54) PSYCHOLOGICAL STATE ANALYSIS METHOD, PSYCHOLOGICAL STATE ANALYSIS APPARATUS AND PROGRAM

(71) Applicant: NIPPON TELEGRAPH AND TELEPHONE CORPORATION, Tokyo (JP)

(72) Inventors: Shuhei YAMAMOTO, Tokyo (JP); Hiroyuki TODA, Tokyo (JP); Takeshi KURASHIMA, Tokyo (JP); Tomu TOMINAGA, Tokyo (JP)

(73) Assignee: NIPPON TELEGRAPH AND TELEPHONE CORPORATION, Tokyo (JP)

(21) Appl. No.: 17/926,607

(22) PCT Filed: May 28, 2020
(86) PCT No.: PCT/JP2020/021081

§ 371 Date: Nov. 20, 2022

(51) Int. Cl. G16H 50/30; A61B 5/16; G06N 3/08

(52) U.S. Cl. CPC G16H 50/30; A61B 5/165; G06N 3/08

(57) ABSTRACT

It is possible to improve the accuracy of estimation of a psychological state of a person by causing a computer to execute calculating probabilities of a plurality of mood transitions based on first time-series data on a mood of a first person in a first period, calculating average persistence times of the plurality of mood transitions based on the first time-series data, and performing learning of a neural network that estimates a psychological state of a person based on a vector including the probabilities of the plurality of mood transitions and a vector including the average persistence times of the plurality of mood transitions, based on the probability calculated for each of the plurality of mood transitions, the average persistence time calculated for each of the plurality of mood transitions, and data indicating, per time interval, the psychological state of the first person in the time interval that is obtained by dividing the first period into a plurality of parts.

### TECHNICAL FIELD

The present disclosure relates to a psychological state analysis method, a psychological state analysis apparatus, and a program.

### BACKGROUND ART

Individual mental health is one indispensable element in the achievement of psychological well-being. The “mood” and “emotion” of an individual are important components in a psychological state (such as a level of depression, a level of stress, or a level of well-being), and changes in these components have an influence on long-term and short-term changes in a psychological state.

For example, long-term negative emotion is one diagnostic criterion for depression, and frequent fluctuations in mood in the short term are a symptom of bipolar disorder. Until now, psychological states have been evaluated through questionnaires designed by doctors, but the burden of an individual's answer to a questionnaire is heavy, and it has not been possible to monitor an individual's psychological state in detail.

However, in recent years, it has become possible to collect individual moods with a high granularity of answer through a simple questionnaire referred to as an ecological momentary assessment (EMA). For example, in an input method referred to as photographic affect meter (PAM), an individual's mood can be recorded by the individual being presented with sixteen images on a smartphone and selecting an image that suits his/her current mood, and thus it is possible to monitor the mood with increased frequency (NPL 1). If the influence of mood on a psychological state can be quantitatively analyzed and predicted according to an individual, it can be useful for the individual to look back on his/her own behavior or for detecting deterioration of the psychological state in advance.

As a related-art technique of analyzing such mood data and data of a psychological state, the development of a technique of performing the regression on a psychological state that is a target variable by using an average value and standard deviation of mood for a specific period as explanatory variables, and a technique of calculating an amount of change in time-series mood and performing the regression on the calculation result have been undertaken (NPL 2).

### CITATION LIST

### Non Patent Literature

NPL 1: J. P. Pollak et al., “PAM: A Photographic Affect Meter for frequent, in situ measurement of affect”, In Proc. of CHI, 2011.

NPL 2: E. Dejonckheere et al., “Complex affect dynamics add limited information to the prediction of psychological well-being”, Journal of Nature Human Behaviour, Vol. 3, pp. 478-491, 2019.

### SUMMARY OF THE INVENTION

### Technical Problem

Unfortunately, in the above methods of the related art, because analysis is performed from the viewpoint of which statistic has a strong influence on the psychological state, it is insufficient in terms of insufficiency of the accuracy of estimation.

The present disclosure is contrived in view of the above points, and an object thereof is to improve the accuracy of estimation of a psychological state of a person.

Means for Solving the Problem

Consequently, in order to solve the above problem, a computer executes a first calculation step of calculating probabilities of a plurality of mood transitions based on first time-series data on a mood of a first person in a first period, a second calculation step of calculating average persistence times of the plurality of mood transitions based on the first time-series data, and a learning step of performing learning on a neural network that estimates a psychological state of a person based on a vector including the probabilities of the plurality of mood transitions and a vector including the average persistence times of the plurality of mood transitions, based on a probability of the probabilities calculated for each of the plurality of mood transitions in the first calculation step, an average persistence time of the average persistence times calculated for each of the plurality of mood transitions in the second calculation step, and data indicating, per time interval, the psychological state of the first person in the time interval that is obtained by dividing the first period into a plurality of parts.

### Effects of the Invention

It is possible to improve the accuracy of estimation of a psychological state of a person.

### BRIEF DESCRIPTION OF DRAWINGS

FIG. 1 is a diagram illustrating a hardware configuration example of a psychological state analysis apparatus 10 according to an embodiment of the present disclosure.

FIG. 2 is a diagram illustrating a functional configuration example of the psychological state analysis apparatus 10 in a learning phase according to the embodiment of the present disclosure.

FIG. 3 is a flowchart illustrating an example of a processing procedure which is executed by the psychological state analysis apparatus 10 in the learning phase.

FIG. 4 is a diagram illustrating a configuration example of a mood data DB 121.

FIG. 5 is a diagram illustrating a configuration example of preprocessed mood data.

FIG. 6 is a diagram illustrating a configuration example of mood transition probability data.

FIG. 7 is a diagram illustrating a configuration example of mood transition time data.

FIG. 8 is a diagram illustrating a configuration example of a psychological state data DB 122.

FIG. 9 is a diagram illustrating a configuration example of preprocessed psychological state data.

FIG. 10 is a diagram illustrating a configuration example of an estimation parameter storage DB 124.

FIG. 11 is a flowchart illustrating an example of a processing procedure for preprocessing of mood data.

FIG. 12 is a flowchart illustrating an example of a processing procedure for processing of generating a mood transition probability data group.

FIG. 13 is a flowchart illustrating an example of a processing procedure for processing of generating a mood transition time data group.

FIG. 14 is a flowchart illustrating an example of a processing procedure for preprocessing of psychological state data.

FIG. 15 is a diagram illustrating an example of a structure of a model according to the present embodiment.

FIG. 16 is a flowchart illustrating an example of a processing procedure for model learning processing.

FIG. 17 is a diagram illustrating a configuration example of model parameters stored in a psychological state estimation model DB 123.

FIG. 18 is a diagram illustrating a functional configuration example of the psychological state analysis apparatus 10 in an estimation phase according to the embodiment of the present disclosure.

FIG. 19 is a flowchart illustrating an example of a processing procedure which is executed by the psychological state analysis apparatus 10 in the estimation phase.

FIG. 20 is a flowchart illustrating an example of a processing procedure for psychological state estimation processing.

FIG. 21 is a flowchart illustrating an example of a processing procedure which is executed by a psychological state data restoration unit 18.

### DESCRIPTION OF EMBODIMENTS

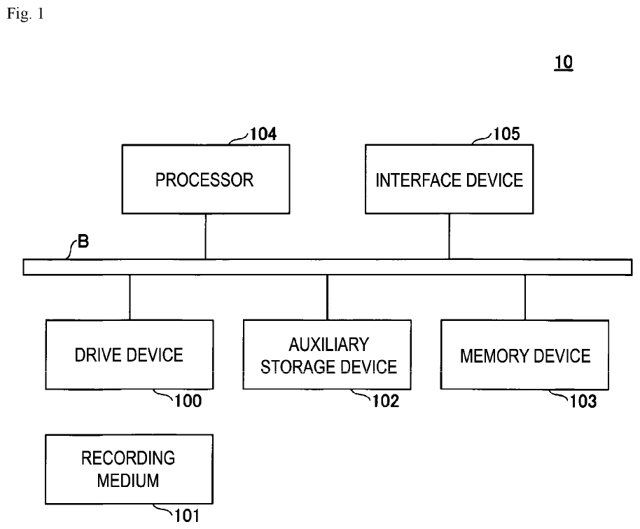

Hereinafter, an embodiment of the present disclosure will be described with reference to the accompanying drawings. FIG. 1 is a diagram illustrating a hardware configuration example of a psychological state analysis apparatus 10 according to the embodiment of the present disclosure. The psychological state analysis apparatus 10 of FIG. 1 includes a drive device 100, an auxiliary storage device 102, a memory device 103, a processor 104, an interface device 105, and the like which are connected to each other through a bus B.

A program for achieving processing in the psychological state analysis apparatus 10 is provided on a recording medium 101 such as a CD-ROM. When the recording medium 101 having a program stored therein is set in the drive device 100, the program is installed from the recording medium 101 through the drive device 100 to the auxiliary storage device 102. However, the program does not necessarily have to be installed from the recording medium 101 and may be downloaded from another computer through a network. The auxiliary storage device 102 stores the installed program and stores necessary files, data, or the like.

In response to an activation instruction of a program, the memory device 103 reads out the program from the auxiliary storage device 102 and stores the program. The processor 104 is a CPU or a graphics processing unit (GPU), or a CPU and a GPU, and executes a function related to the psychological state analysis apparatus 10 in accordance with the program stored in the memory device 103. The interface device 105 is used as an interface for connection to a network.

In the present embodiment, processing which is executed by the psychological state analysis apparatus 10 is classified into a learning phase and an estimation phase. First, the learning phase will be described.

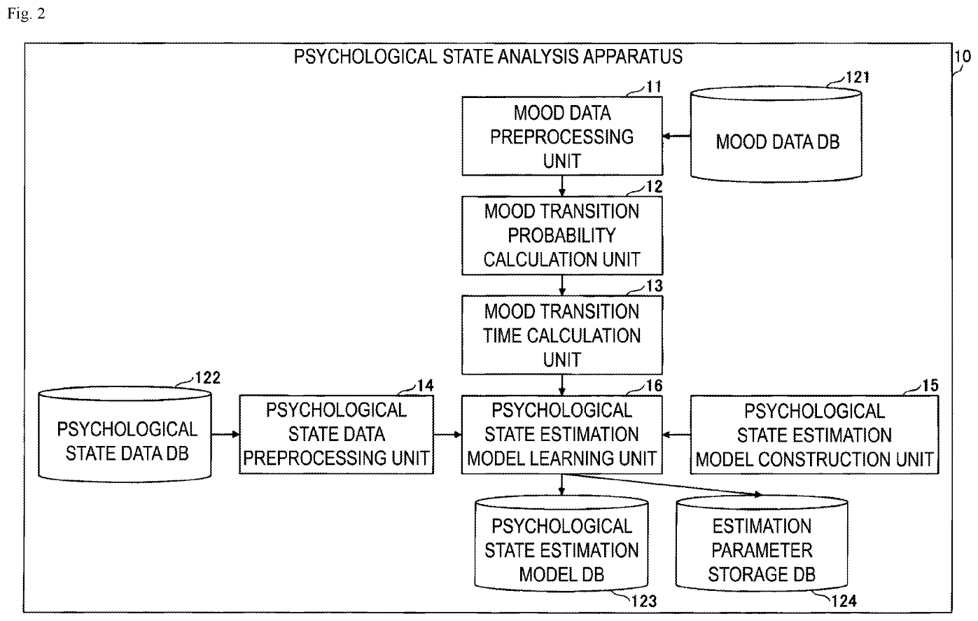

FIG. 2 is a diagram illustrating a functional configuration example of the psychological state analysis apparatus 10 in the learning phase according to the embodiment of the present disclosure. As illustrated in FIG. 2, the psychological state analysis apparatus 10 in the learning phase includes a mood data preprocessing unit 11, a mood transition probability calculation unit 12, a mood transition time calculation unit 13, a psychological state data preprocessing unit 14, a psychological state estimation model construction unit 15, a psychological state estimation model learning unit 16, and the like. Each of these units is implemented by one or more programs installed in the psychological state analysis apparatus 10 causing the processor 104 to execute processing. In addition, the psychological state analysis apparatus 10 in the learning phase uses databases (storage units) such as a mood data DB 121, a psychological state data DB 122, a psychological state estimation model DB 123, and an estimation parameter storage DB 124. Each of these databases can be implemented by using, for example, a storage device or the like that can be connected to the auxiliary storage device 102 or the psychological state analysis apparatus 10 through a network.

The mood data DB 121 stores character strings expressing the moods of a certain person (hereinafter referred to as a “user A”) at a plurality of timings in a certain period (hereinafter referred to as a “period Ti”) together with indicating each timing. Meanwhile, the mood of the user A may be acquired by, for example, the self-report of the user A.

The psychological state data DB 122 stores numerical values or character strings indicating the psychological states of the user A at a plurality of timings in a period T2 obtained by the user A answering to a questionnaire at the plurality of timings. Meanwhile, the cycle of timing at which the psychological state of the user A is acquired is longer than the cycle at which the mood of the user A is acquired.

Regarding the construction of the mood data DB 121 or the psychological state data DB 122, for example, a score or a character string obtained to be a result of the user A answering to a questionnaire may be input, and the input result may be stored in the DB together with the answer time.

The psychological state analysis apparatus 10 in the learning phase uses each database (DB) to perform learning on a neural network as a psychological state estimation model for estimating a psychological state (hereinafter simply referred to as a “model”) and outputs a parameter (α to be described below) unique to learning data revealed in the learning of the model.

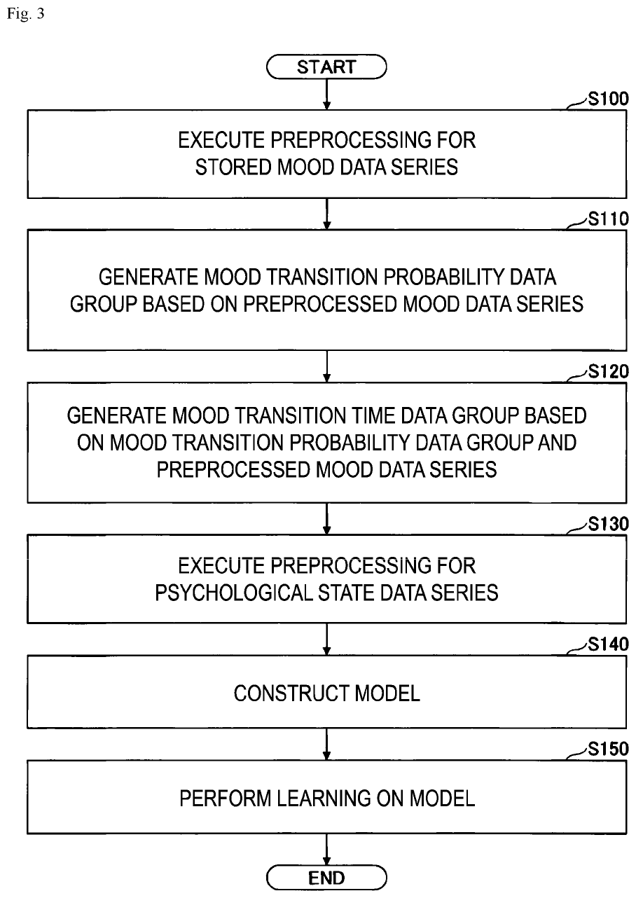

FIG. 3 is a flowchart illustrating an example of a processing procedure which is executed by the psychological state analysis apparatus 10 in the learning phase.

In step S100, the mood data preprocessing unit 11 executes preprocessing for each piece of data stored in the mood data DB 121 in a time series (hereinafter referred to as “mood data”). The details of preprocessing will be described below.

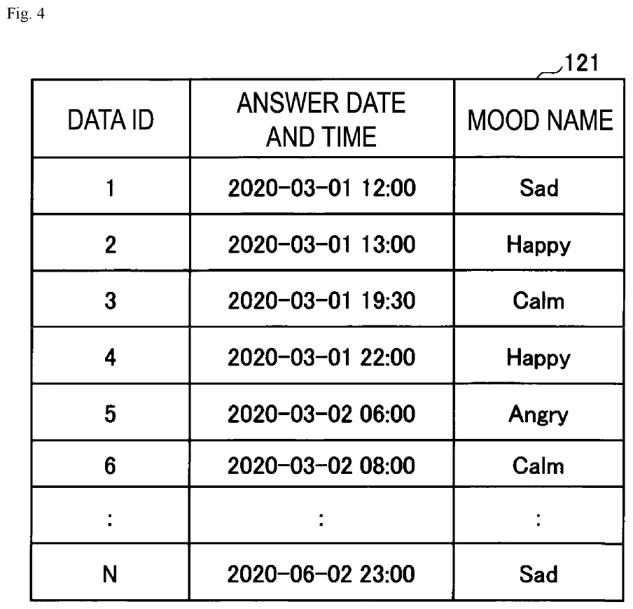

FIG. 4 is a diagram illustrating a configuration example of the mood data DB 121. As illustrated in FIG. 4, the mood data DB 121 stores one or more mood data series in a time-series order. The mood data includes a data ID, an answer date and time, a mood name, and the like. The data ID is identification information of the mood data. The answer date and time is an answer date and time of the mood of the user A (that is, a date and time when the user A is in a mood indicated by the mood name). The mood name is a character string indicating a mood. Meanwhile, the mood data is recorded in the mood data DB 121, for example, at a timing when the mood changes. In this case, the mood name of the recorded mood data is a mood name after the change. However, the mood data may be recorded at a timing of a fixed cycle such as an hour interval or at any timing.

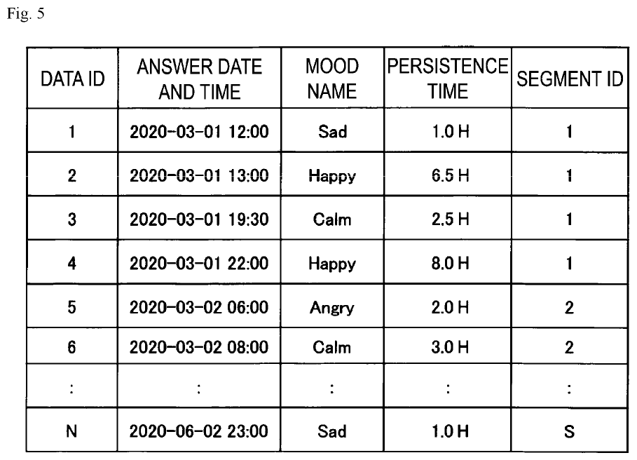

In step S100, a series of data (hereinafter referred to as “preprocessed mood data”) as illustrated in FIG. 5 is generated based on a series of the mood data illustrated in FIG. 4.

FIG. 5 is a diagram illustrating a configuration example of preprocessed mood data. One row in FIG. 5 is equivalent to one piece of preprocessed data. As illustrated in FIG. 5, the preprocessed mood data includes a persistence time and a segment ID in addition to the mood data. The persistence time is a time for which a mood indicated by the mood name continues. The segment ID is an ID which is assigned based on a predetermined rule to be described below and is equivalent to identification information for each time interval in which the period T1 is divided into a plurality of parts.

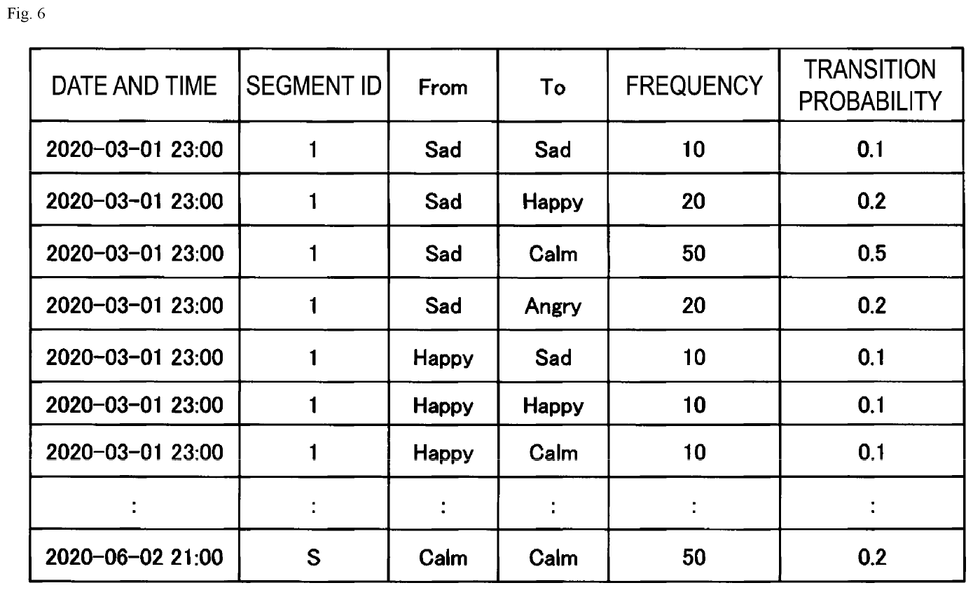

Next, the mood transition probability calculation unit 12 receives preprocessed mood data series from the mood data preprocessing unit 11 and calculates a transition probability for each of the plurality of mood transitions based on the preprocessed mood data series (S110). As a result, a group of data illustrated in FIG. 6 (hereinafter referred to as “mood transition probability data”) is generated. Meanwhile, the details of generation of a mood transition probability data group will be described below.

FIG. 6 is a diagram illustrating a configuration example of mood transition probability data. One row in FIG. 6 is equivalent to one piece of mood transition probability data.

Next, the mood transition time calculation unit 13 receives a mood transition probability data group and a preprocessed mood data series from the mood transition probability calculation unit 12 and generates a mood transition time data group based on the mood transition probability data group and the preprocessed mood data series (S120). Meanwhile, the details of generation of the mood transition time data group will be described below.

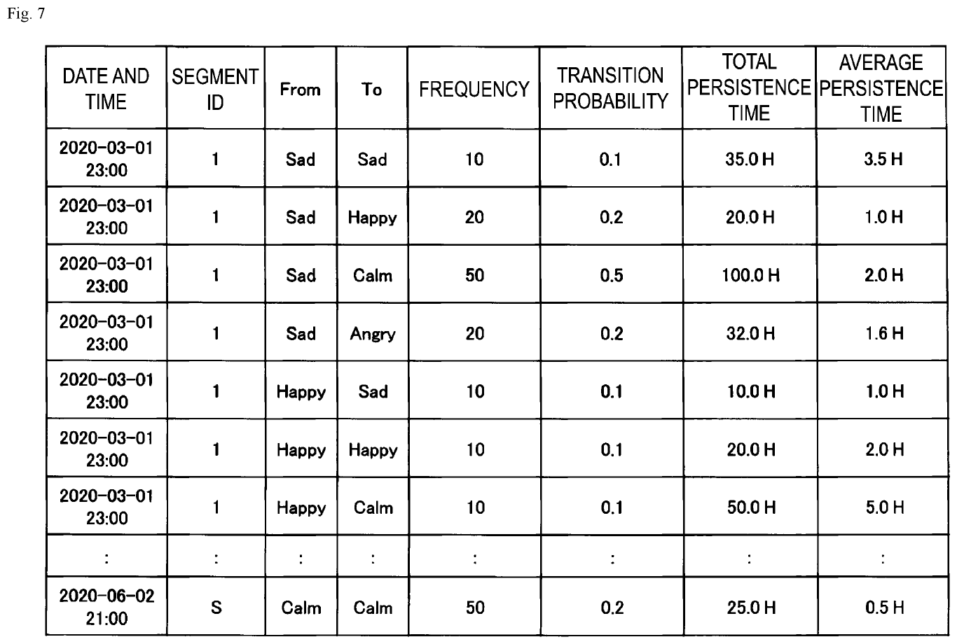

FIG. 7 is a diagram illustrating a configuration example of mood transition time data. One row in FIG. 7 is equivalent to one piece of mood transition time data. As illustrated in FIG. 7, the mood transition time data is data in which “total persistence time” and “average persistence time” are assigned to the mood transition probability data. Meanwhile, the mood transition time data may not include “frequency” and “transition probability”.

Next, the psychological state data preprocessing unit 14 executes preprocessing for a series of psychological state data stored in the psychological state data DB 122 (S130). The details of the preprocessing will be described below.

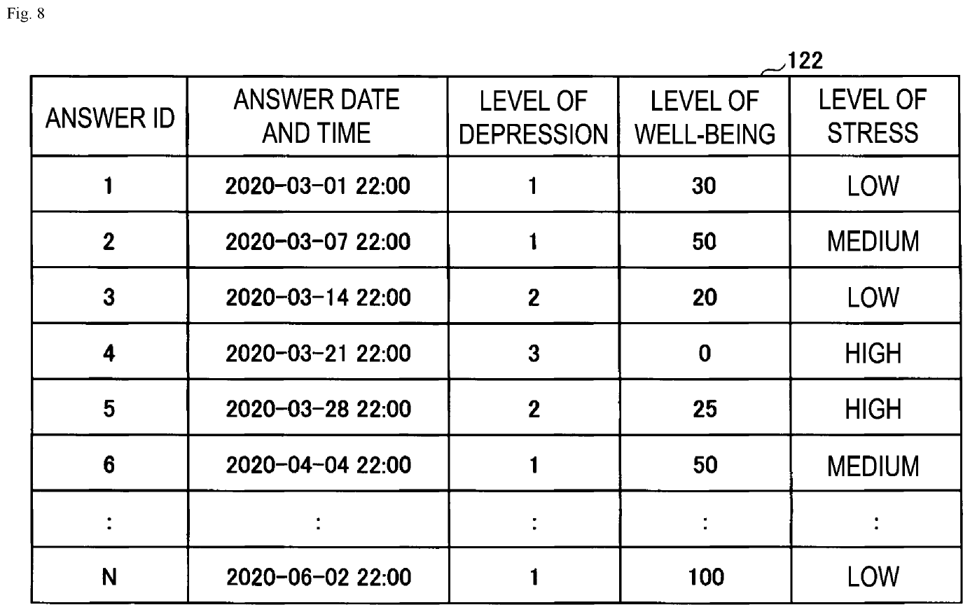

FIG. 8 is a diagram illustrating a configuration example of the psychological state data DB 122. One row in FIG. 8 is equivalent to one piece of psychological state data. The psychological state data DB 122 stores psychological state data in a time-series order. The psychological state data includes an answer ID, an answer date and time, a level of depression, a level of well-being, a level of stress, and the like. The answer ID is identification information of an answer of the user A to a questionnaire. The questionnaire is composed of, for example, a plurality of questions. The level of depression, the level of well-being, and the level of stress are examples of indexes indicating a psychological state which are derived based on answers to a plurality of questions included in a questionnaire. Thus, one answer to a questionnaire corresponds to one piece of psychological state data. That is, it can be said that the answer ID is identification information of the psychological state data. The answer date and time is a date and time when an answer to a questionnaire is performed. In the present embodiment, an example in which questionnaires are performed at one week intervals in the period T1 is illustrated. Thus, the interval of the answer date and time of the psychological state data is one week. The level of depression is a score indicating the level of depression obtained based on an answer to a questionnaire. The level of well-being is a score indicating the level of feeling of well-being obtained based on the answer. The level of stress is a score (low, medium, high) indicating the level of stress obtained based on the answer. Meanwhile, the output of one model is any one of the level of depression, the level of well-being, and the level of stress. Thus, only one of these indexes may be included in the psychological state data.

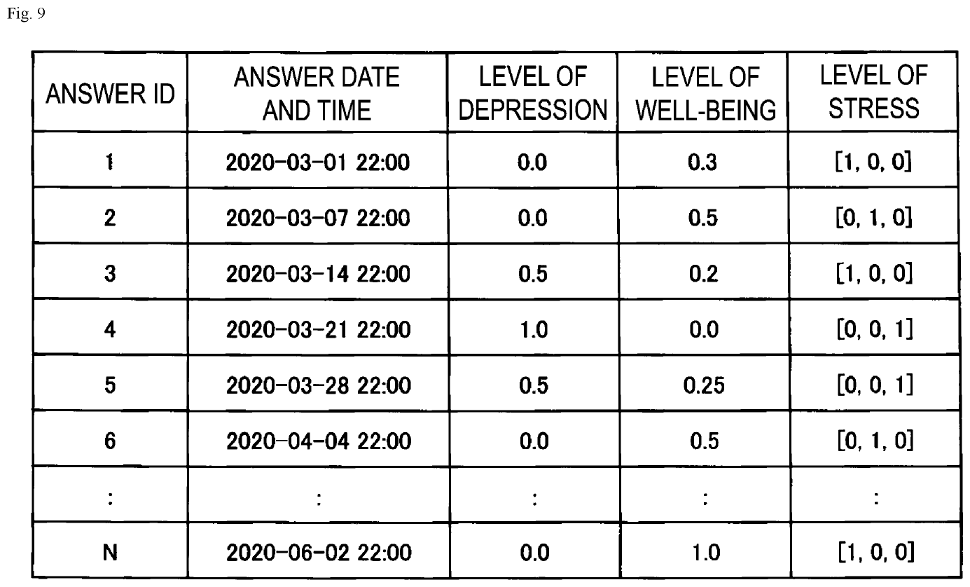

In step S130, a series of data (hereinafter referred to as “preprocessed psychological state data”) as illustrated in FIG. 9 is generated based on the psychological state data series illustrated in FIG. 8.

FIG. 9 is a diagram illustrating a configuration example of preprocessed psychological state data. One row in FIG. 9 is equivalent to one piece of preprocessed psychological state data. As illustrated in FIG. 9, items included in the preprocessed psychological state data are the same as those in the psychological state data. However, the values of the level of depression, the level of well-being, and the level of stress are converted.

Next, the psychological state estimation model construction unit 15 constructs a model (S140). The construction of a model involves, for example, loading a program as a model into the memory device 103. Meanwhile, the details of the structure of the model will be described below.

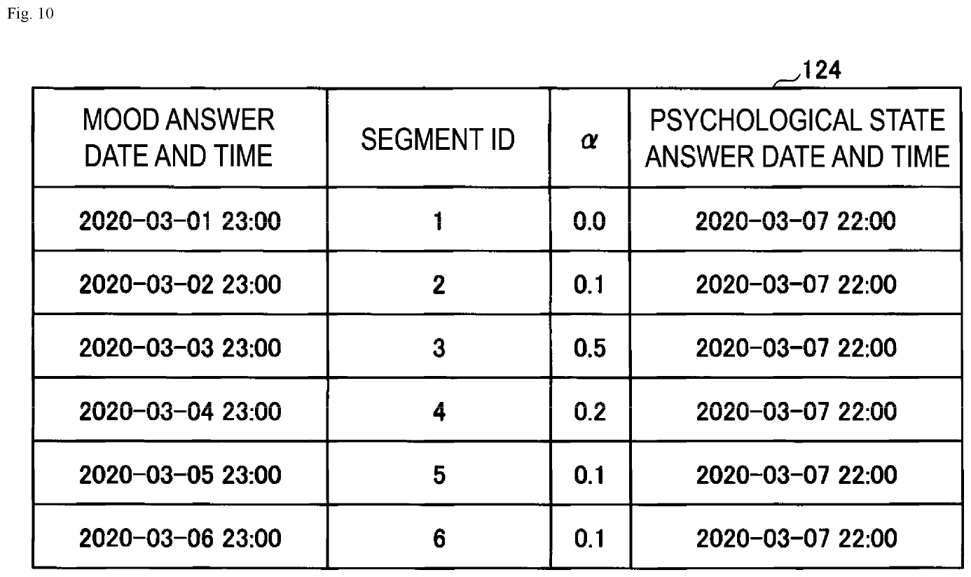

Next, the psychological state estimation model learning unit 16 receives the mood transition probability data group (FIG. 6) and the mood transition time data group (FIG. 7) from the mood transition time calculation unit 13, receives the preprocessed psychological state data series (FIG. 9) from the psychological state data preprocessing unit 14, receives the model from the psychological state estimation model construction unit 15, and performs learning on the model (S150). The psychological state estimation model learning unit 16 outputs model parameters of the learned model to the psychological state estimation model DB 123 and outputs a parameter (α to be described below) obtained in a learning process to the estimation parameter storage DB 124. FIG. 10 illustrates a configuration example of the estimation parameter storage DB 124. Meanwhile, the details of model learning will be described below.

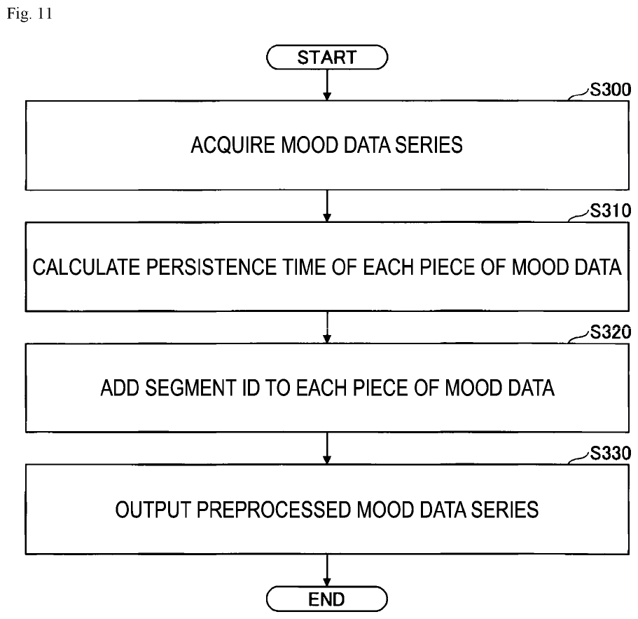

Next, the details of step S100 will be described. FIG. 11 is a flowchart illustrating an example of a processing procedure for preprocessing of mood data.

In step S300, the mood data preprocessing unit 11 acquires a mood data series. In the learning phase, the mood data series stored in the mood data DB 121 is acquired.

Next, the mood data preprocessing unit 11 calculates a persistence time for each piece of mood data included in the acquired mood data series (S310). Specifically, the mood data preprocessing unit 11 scans the mood data series in ascending order of the answer date and time, calculates a difference in the answer date and time from the next mood data for each piece of mood data, and stores the difference in the item (column) of the persistence time of the preprocessed mood data corresponding to each piece of mood data. For example, because the difference in the answer date and time between the data ID: 1 and the data ID: 2 is one hour, “1.0 H” is stored in the item (column) of the persistence time of the preprocessed mood data of the data ID: 1.

Next, the mood data preprocessing unit 11 assigns a segment ID to each piece of mood data included in the mood data series (S320). That is, each piece of mood data is classified for each time interval in which the period T1 is divided into a plurality of parts. The segment ID is assigned in accordance with, for example, a rule which is set in advance by a system manager. The rule may be, for example, any of the following three types. Meanwhile, in either case, the segment ID starts from 1, and the same rule is applied to all the mood data included in the mood data series.

Rule 1: The same segment ID is assigned up to preprocessed mood data of which the persistence time exceeds an appropriate threshold. The threshold is set in advance by, for example, a system manager. In a case where the persistence time of certain preprocessed mood data exceeds the threshold, 1 is added to the segment ID, and the segment ID to which 1 has been added is assigned to the certain preprocessed mood data and the subsequent data. For example, in a case where the threshold is set to a value close to the sleep time, sleep can be used as a time interval delimiter.

Rule 2: The same segment ID is assigned until the date changes. In a case where the date changes in certain preprocessed mood data, 1 is added to the segment ID, and the segment ID to which 1 has been added is assigned to the certain preprocessed mood data and the subsequent data.

Rule 3: The same segment ID is assigned until the week changes.

Meanwhile, the mood data preprocessing unit 11 stores the segment ID assigned to each piece of mood data in the item (column) of the segment ID of the preprocessed mood data corresponding to each piece of mood data.

Next, the mood data preprocessing unit 11 outputs the preprocessed mood data series generated corresponding to the mood data series to the mood transition probability calculation unit 12 (S330).

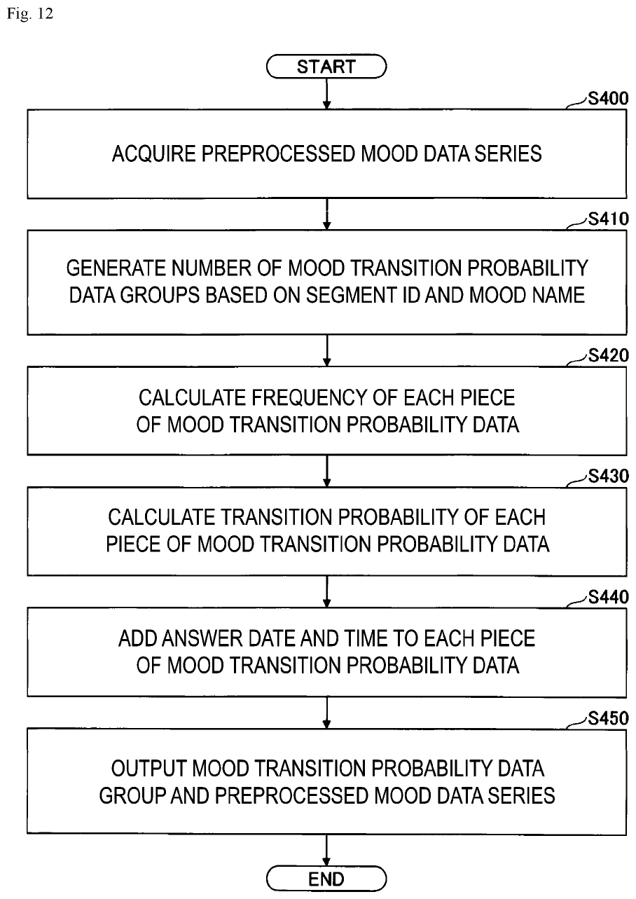

Next, the details of step S110 in FIG. 3 will be described. FIG. 12 is a flowchart illustrating an example of a processing procedure for processing of generating a mood transition probability data group.

In step S400, the mood transition probability calculation unit 12 acquires the preprocessed mood data series output from the mood data preprocessing unit 11.

Next, the mood transition probability calculation unit 12 generates the number of mood transition probability data groups based on combinations of the segment IDs and the mood names of the preprocessed mood data series (S410). Specifically, the mood transition probability calculation unit 12 scans the columns of the segment ID and the mood name of the preprocessed mood data series, specifies the maximum value of the segment ID, and counts the number of types of mood names. The mood transition probability calculation unit 12 generates mood transition probability data for each of the segment IDs and for each of the combinations of two types of mood names. All the combinations of two types of mood names also include sets with the same mood name for both. In addition, sets with a different order of mood names are considered to be different sets. For example, {Sad, Happy} and {Happy, Sad} are considered to be different sets. Thus, for example, when the maximum value of segment ID is S and the number of types of mood names is M, the number of pieces of mood transition probability data to be generated is S×M×M. Meanwhile, FIG. 6 illustrates an example in which there are four types of mood names. Thus, sixteen mood transition probability data groups are generated for one segment ID.

The mood transition probability calculation unit 12 stores the segment ID corresponding to the mood transition probability data in the “segment ID” of each piece of generated mood transition probability data, stores a first mood name of the combination of mood names corresponding to the mood transition probability data in “from”, and stores a second mood name of the combination of mood names corresponding to the mood transition probability data in “to”.

Next, the mood transition probability calculation unit 12 calculates the value of “frequency” of each piece of mood transition probability data and stores the calculation result in the column of the “frequency” (S410). Specifically, the mood transition probability calculation unit 12 scans the preprocessed mood data series in the order of the answer date and time and acquires the column of the “mood name” of two consecutive pieces of preprocessed mood data by segment ID. The mood transition probability calculation unit 12 counts, for each type of the acquired column, the number of appearances of the column corresponding to the type by segment ID. The mood transition probability calculation unit 12 stores, for each segment ID, the count result of the number of appearances of the type of column in the “frequency” of the mood transition probability data that is the mood transition probability data including the segment ID and where the first mood name of the column of mood names acquired in relation to the segment ID matches “from” and the second mood name of the column of mood names acquired in relation to the segment ID matches “to”.

Next, the mood transition probability calculation unit 12 calculates the value of the “transition probability” of each piece of mood transition probability data and stores the calculation result in the column of the “transition probability” (S430). Specifically, the mood transition probability calculation unit 12 calculates, for each segment ID, the total value of the values of the “frequency” of the mood transition probability data including the segment ID and divides the value of the “frequency” by the total value to calculate the “transition probability” of the mood transition probability data.

Next, the mood transition probability calculation unit 12 specifies, for each segment ID, the latest answer date and time among the answer dates and times of the preprocessed mood data series including the segment ID and stores the specified answer date and time in the “answer date and time” of each piece of mood transition probability data including the segment ID (S440).

Next, the mood transition probability calculation unit 12 outputs the generated mood transition probability data group (FIG. 6) and the preprocessed mood data series (FIG. 5), which is acquired in step S400, to the mood transition time calculation unit 13 (S450).

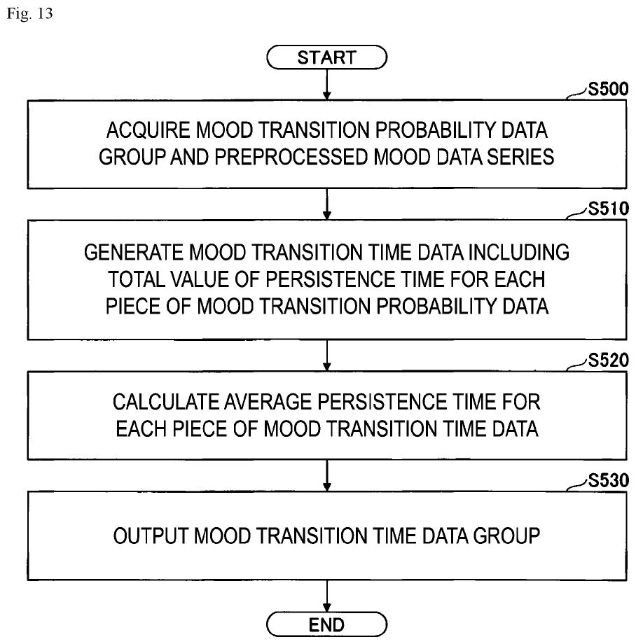

Next, the details of step S120 in FIG. 3 will be described. FIG. 13 is a flowchart illustrating an example of a processing procedure for processing of generating a mood transition time data group.

In step S500, the mood transition time calculation unit 13 acquires the mood transition probability data group and the preprocessed mood data series that are output from the mood transition probability calculation unit 12.

Next, the mood transition time calculation unit 13 scans the preprocessed mood data series, calculates the total value of the “persistence time” for each piece of mood transition probability data (that is, for each “segment ID”, “from”, and “to”), and generates mood transition time data (FIG. 7) including the calculation result (“total persistence time”) and the mood transition probability data (S510). However, at this point in time, the value of “average persistence time” of each piece of mood transition time data is empty.

Specifically, the mood transition time calculation unit 13 calculates, by segment ID, the total value of the persistence time for each type of the mood name column of two consecutive pieces of preprocessed mood data in the preprocessed mood data series. In this case, the persistence time of the first preprocessed mood data out of the two consecutive pieces of preprocessed mood data is added to the total value of the persistence time of the columns of two mood names related to the two pieces of preprocessed mood data. As a result, the total value of the persistence time is calculated for each segment ID and for each type of mood name column. The mood transition time calculation unit 13 generates, for each piece of mood transition probability data of the mood transition probability data group, the mood transition time data including the total value of the persistence time calculated for “segment ID”, “from”, and “to” of the mood transition probability data as “total persistence time” and the mood transition probability data. In this case, “from” corresponds to the first mood name in the mood name column, and “to” corresponds to the second mood name in the mood name column.

Next, the mood transition time calculation unit 13 calculates the “average persistence time” for each piece of mood transition time data and stores the calculation result in the “average persistence time” of the mood transition time data (S520). Specifically, the mood transition time calculation unit 13 calculates the “average persistence time” of the mood transition time data by dividing the “total persistence time” of the mood transition time data by the “frequency” of the mood transition time data.

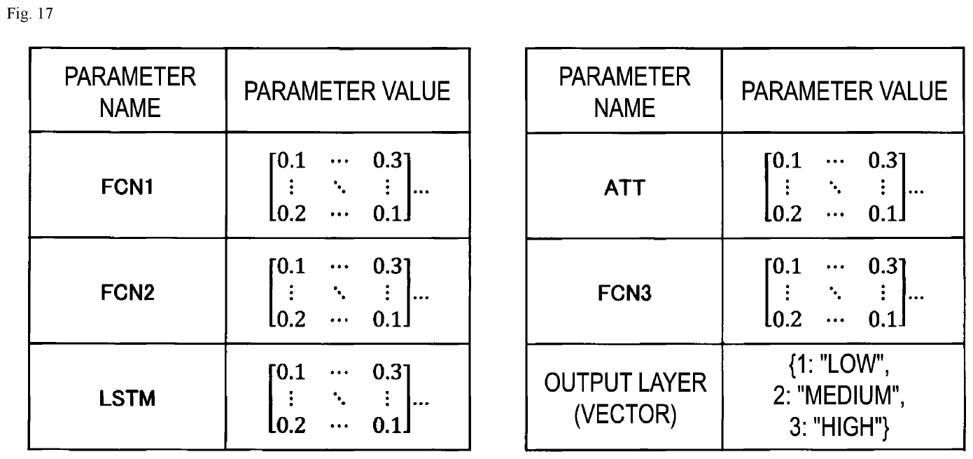

Next, the mood transition time calculation unit 13 outputs the generated mood transition time data group (FIG. 17) to the psychological state estimation model learning unit 16 (S530).

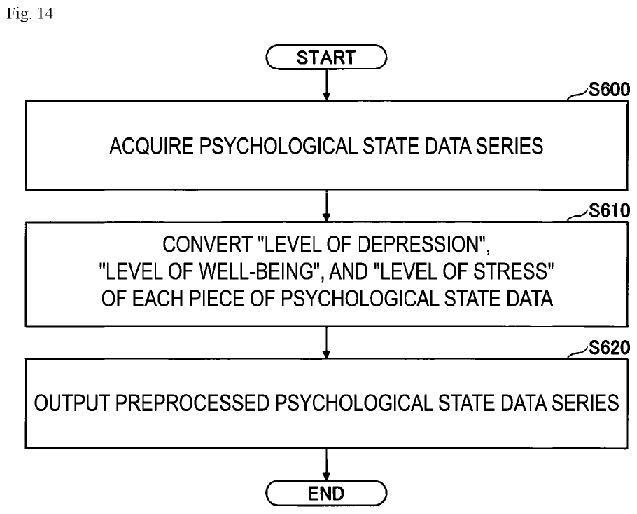

Next, the details of step S130 in FIG. 3 will be described. FIG. 14 is a flowchart illustrating an example of a processing procedure for preprocessing of psychological state data.

In step S600, the psychological state data preprocessing unit 14 acquires a series of all psychological state data (FIG. 8) stored in the psychological state data DB 122.

Next, the psychological state data preprocessing unit 14 converts the values of the items of “level of depression”, “level of well-being”, and “level of stress” of each piece of psychological state data in accordance with the following conditions (S610).

Condition 1: For items of which the values are real numbers (scalar values) (“level of depression” and “level of well-being” in the present embodiment), the maximum value max and the minimum value min among the values of the items in the psychological state data series are specified, and the value x of the item of each piece of psychological state data is converted into x′ in accordance with the following equation.

x′=(x−min)/(max−min)

Condition 2: For an item of which the column value is a character string (“level of stress” in the present embodiment), the number of types of values (character strings) of the item in the psychological state data series is counted, and the value of the item of each piece of psychological state data is converted into a vector of one-hot expression with the number of types as the vector size.

Next, the psychological state data preprocessing unit 14 outputs the preprocessed psychological state data series (FIG. 9) after conversion of the value of each of the items above to the psychological state estimation model learning unit 16 (S620).

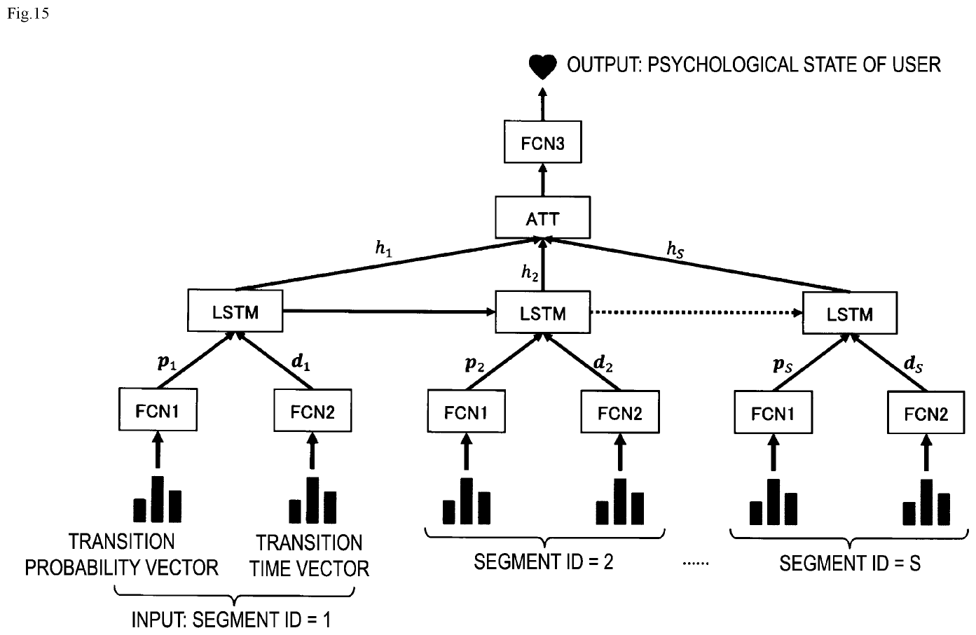

Next, the details of the structure of a model which is constructed in step S140 of FIG. 3 will be described. FIG. 15 is a diagram illustrating an example of the structure of a model according to the present embodiment. As illustrated in FIG. 15, the model of the present embodiment has a structure of a deep neural network (DNN).

The model of the present embodiment receives, as inputs, a transition probability vector and a transition time vector for each segment ID and outputs the estimation value of a psychological state. Here, the transition probability vector is a vector having a set of transition probabilities of each of a plurality of pieces of mood transition probability data including the same segment ID as one element. When the number of types of mood names is M, the number of pieces of mood transition probability data for one segment ID is M×M, and thus the number of elements of one transition probability vector is M×M.

In addition, the transition time vector is a vector having a set of average persistence times of each of a plurality of pieces of mood transition time data including the same segment ID as one element. When the number of types of mood names is M, the number of pieces of mood transition time data for one segment ID is M×M, and thus the number of elements of one transition time vector is M×M.

The output estimation value of a psychological state is an estimation value of the index of any one of the level of depression, the level of well-being, and the level of stress in the present embodiment. In a case where the index corresponds to the above condition 1 (scalar value), the scalar value is an estimation value of a psychological state. In a case where the index corresponds to the above condition 2 (character string), the probability value for each character string is an estimation value of a psychological state.

In FIG. 15, the model includes the following five units.

The first unit is a fully connected layer 1 (FCN 1) that extracts a more abstract feature from the transition probability vector. The FCN 1 uses, for example, a sigmoid function, a ReLu function, or the like to perform non-linear transformation on the feature amount of input (the transition probability vector) and obtains a feature vector ps. Where, s represents an index related to the segment ID.

The second unit is a fully connected layer 2 (FCN 2) that extracts a more abstract feature from the transition time vector. The FCN 2 uses, for example, a sigmoid function, a ReLu function, or the like to perform non-linear transformation on the feature amount of input (the transition time vector) and obtains a feature vector dₛ. Where, s represents an index related to the segment ID.

The third unit is a long-short term memory (LSTM) that further abstracts the abstracted feature vectors pₛ and dₛ as a series feature vector {hₛ}ₛ=1^{s}. Specifically, the LSTM sequentially receives the series of pₛ and dₛ and repeatedly performs non-linear transformation while considering the past abstracted feature vector hₛ. Meanwhile, in FIG. 15, the LSTM is illustrated expanded in a time series.

The fourth unit is a self-attention mechanism (ATT) that obtains a feature vector considering the degree of each importance for the series feature vector {hₛ}ₛ=1^{s} abstracted by the LSTM. A weight {αₛ}ₛ=1^{s} for considering each feature vector is obtained by two fully connected layers. The first fully connected layer outputs a context vector of any size with hₛ as an input, and the second fully connected layer outputs a scalar value corresponding to the importance as with the context vector as an input. The context vector may undergo non-linear transformation processing. The importance αₛ is converted into a value corresponding to a probability value by, for example, a softmax function or the like.

The fifth unit is a fully connected layer 3 (FCN 3) that calculates the value of one of indexes indicating the psychological states of the user A (a scalar value or a probability vector of the dimension of the number of types of psychological states) by using a feature vector weighted averaged by a self-attention mechanism (ATT). In a case where the output is a probability vector, a non-linear transformation is performed so that the sum of all elements of output feature amounts is set to 1 by using a softmax function or the like.

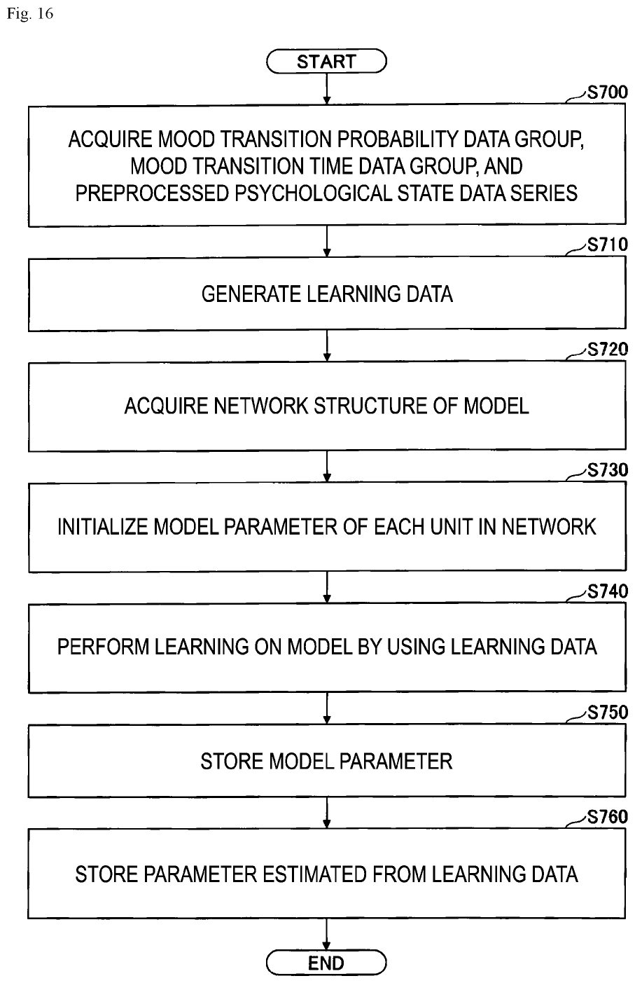

Next, the details of step S150 in FIG. 3 will be described. FIG. 16 is a flowchart illustrating an example of a processing procedure for model learning processing.

In step S700, the psychological state estimation model learning unit 16 acquires the mood transition probability data group (FIG. 6) and the mood transition time data group (FIG. 7) output from the mood transition time calculation unit 13 and the preprocessed psychological state data series (FIG. 9) output from the psychological state data preprocessing unit 14.

Next, the psychological state estimation model learning unit 16 generates learning data for the model (S710). Specifically, the psychological state estimation model learning unit 16 first generates, for each segment ID, a group constituted by the mood transition probability data (FIG. 6) including the segment ID and the mood transition time data (FIG. 7) including the segment ID. When the number of types of mood names is M, one group includes M×M pieces of mood transition probability data and M x M pieces of mood transition time data. The mood transition probability data and the mood transition time data of each group are equivalent to input values in the learning data.

In addition, the psychological state estimation model learning unit 16 associates each piece of preprocessed psychological state data (FIG. 9) with a group to which the mood transition time data (FIG. 7) whose “date and time” is included in a period between the “answer date and time” of the preprocessed psychological state data and the “answer date and time” of the immediately preceding preprocessed psychological state data belongs. For example, in a case where one segment ID corresponds to one day and the psychological state data is recorded at one week intervals, seven groups are associated with one piece of preprocessed psychological state data. The preprocessed psychological state data associated with a group is equivalent to the output value in the learning data.

Next, the psychological state estimation model learning unit 16 acquires the network structure of the model as illustrated in FIG. 15 from the psychological state estimation model construction unit 15 (S720).

Next, the psychological state estimation model learning unit 16 initializes the model parameters of each unit in the network related to the network structure (S730). For example, each model parameter is initialized with a random number between 0 and 1.

Next, the psychological state estimation model learning unit 16 performs learning on the model by using the above learning data (S740). As a result of learning, the model parameters are updated. More specifically, the psychological state estimation model learning unit 16 updates the model parameters based on the comparison between a value which is output from the model by inputting a transition probability vector series and a transition time vector series based on a plurality of groups associated with the same preprocessed psychological state data into the model and a value of the index of one psychological state that is an estimation target in the preprocessed psychological state data. Such updating is performed in the time-series order of the preprocessed psychological state data series.

Meanwhile, the transition probability vector based on a group is a transition probability vector based on the mood transition probability data group included in a group that belongs to the group, and the transition time vector based on a group is a transition time vector based on the mood transition time data group included in a group that belongs to the group. By arranging the transition probability vectors and the transition time vectors of each of a plurality of groups associated with the same preprocessed psychological state data in ascending order of the segment ID corresponding to each of these groups, a transition probability vector series and a transition time vector series corresponding to the preprocessed psychological state data are obtained.

Next, the psychological state estimation model learning unit 16 stores the model parameters of the learned model in the psychological state estimation model DB 123 (S750).

FIG. 17 is a diagram illustrating a configuration example of model parameters stored in the psychological state estimation model DB 123. As illustrated in FIG. 17, the model parameters in each layer are stored in the psychological state estimation model DB 123 as a matrix or a vector. In addition, the text of a psychological state corresponding to each element number of the probability vector is stored in the output layer. Meanwhile, the output layer corresponds to an example in which the index of the psychological state to be estimated is “level of stress”.

Next, the psychological state estimation model learning unit 16 stores the parameter {αₛ}ₛ=1^{s} estimated (calculated) based on the learning data in the estimation parameter storage DB 124 (FIG. 10) in association with the segment ID and the date and time of each group of the learning data (S760). Meanwhile, the parameters stored in the estimation parameter storage DB 124 have significance as reference information during learning and are not used in the estimation phase. Thus, step S760 may not be executed.

Meanwhile, in the above, although an example in which the learning of the model is performed using only the mood data series and psychological state data series of one person (the user A) is illustrated, the learning may be performed using these data series of a plurality of persons. That is, one model may be learned for each individual, or one model may be learned for a plurality of persons.

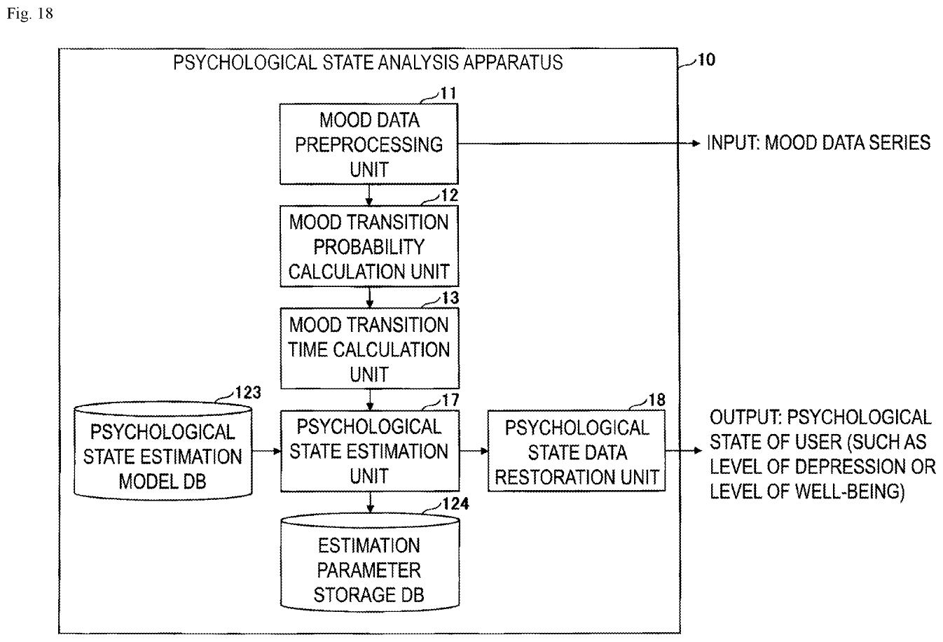

Next, the estimation phase will be described. FIG. 18 is a diagram illustrating a functional configuration example of the psychological state analysis apparatus 10 in the estimation phase according to the embodiment of the present disclosure. In FIG. 18, the same parts as those in FIG. 2 or the corresponding parts are designated by the same reference numerals.

As illustrated in FIG. 18, the psychological state analysis apparatus 10 in the estimation phase includes the mood data preprocessing unit 11, the mood transition probability calculation unit 12, the mood transition time calculation unit 13, a psychological state estimation unit 17, a psychological state data restoration unit 18, and the like. Each of these units is implemented by one or more programs installed in the psychological state analysis apparatus 10 causing the processor 104 to execute processing. In addition, the psychological state analysis apparatus 10 in the estimation phase uses the psychological state estimation model DB 123 and the estimation parameter storage DB 124.

The psychological state analysis apparatus 10 in the estimation phase outputs the estimation result of the psychological state for the input mood data series and the parameter a obtained during the estimation as the analyze result.

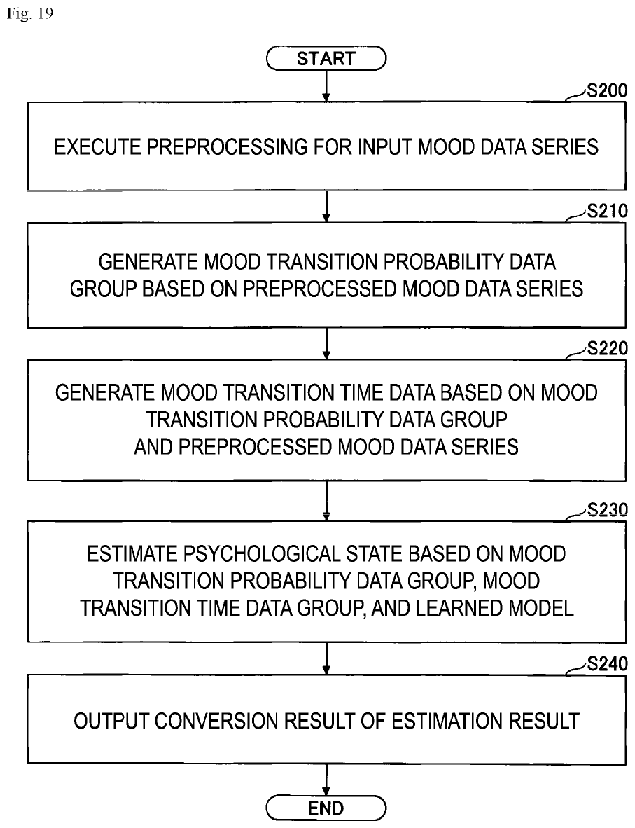

FIG. 19 is a flowchart illustrating an example of a processing procedure which is executed by the psychological state analysis apparatus 10 in the estimation phase.

In step S200, the mood data preprocessing unit 11 executes the preprocessing described in FIG. 11 on a mood data series of a certain person (hereinafter referred to as a “user B”) in a certain period (hereinafter referred to as a “period T2”) that is input as an estimation target. However, the mood data series that is acquired in step S300 of FIG. 11, which is executed in connection with step S200, is the input mood data series. As a result, the preprocessed mood data series based on the mood data series is generated. Meanwhile, the user B may be the same person as the user A, or may be a person different from the user A. In addition, in a case where the user B is a person different from the user A, the period T2 may be the same period as the period T1.

Next, the mood transition probability calculation unit 12 receives the preprocessed mood data series from the mood data preprocessing unit 11 and executes the processing described in FIG. 12 on the preprocessed mood data series (S210). As a result, the mood transition probability data group based on the preprocessed mood data series is generated.

Next, the mood transition time calculation unit 13 receives the mood transition probability data group from the mood transition probability calculation unit 12, receives the preprocessed mood data series from the mood data preprocessing unit 11, and executes the processing described in FIG. 13 on the mood transition probability data group and the preprocessed mood data series (S220). As a result, the mood transition probability data group is generated based on the mood transition probability data group and the preprocessed mood data series.

Next, the psychological state estimation unit 17 receives the mood transition probability data group and the mood transition time data group from the mood transition time calculation unit 13, acquires the learned model from the psychological state estimation model DB 123, and estimates (calculates) the psychological state of the user BTW in the period T2 (S230). The psychological state estimation unit 17 outputs the value of the parameter a obtained in the calculation processing to the estimation parameter storage DB 124.

Next, the psychological state data restoration unit 18 receives the estimation result from the psychological state estimation unit 17 and outputs the conversion result of the estimation result (S240). The details of the processing will be described below.

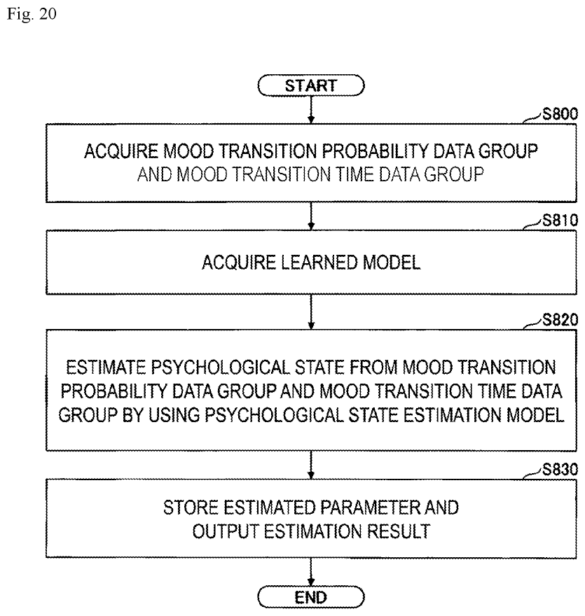

Next, the details of step S230 will be described. FIG. 20 is a flowchart illustrating an example of a processing procedure for psychological state estimation processing.

In step S800, the psychological state estimation unit 17 acquires the mood transition probability data group and the mood transition probability data group which are output from the mood transition time calculation unit 13.

Next, the psychological state estimation unit 17 acquires the learned model from the psychological state estimation model DB 123 (S810).

Next, the psychological state estimation unit 17 calculates the estimation value (probability value or scalar value) of the index of the psychological state for each segment ID by inputting the mood transition probability data group and the mood transition time data group to the learned model (S820). More specifically, similarly to the case of learning, the psychological state estimation unit 17 generates a transition probability vector and a transition time vector for each segment ID based on the mood transition probability data group and the mood transition time data group and inputs a series of the generated transition probability vector and a series of the generated transition time vector to the learned model. As a result, the estimation value of the psychological state of the user B in the period T2 is output from the learned model. In this processing, the psychological state estimation unit 17 estimates (calculates) the parameter {αₛ}ₛ=1^{s} by using the self-attention mechanism (ATT) of the model. That is, as is estimated (calculated) for each segment ID (for each time interval in which the period T2 is divided into a plurality of parts).

Next, the psychological state estimation unit 17 stores each of the estimated (calculated) parameters {αₛ}ₛ=1^{s} in the estimation parameter storage DB 124 in association with the segment ID (S830). In addition, the psychological state estimation unit 17 outputs the estimation result (the estimation value of the psychological state) of the learned model to the psychological state data restoration unit 18. Meanwhile, each a which is associated with the segment ID and stored in the estimation parameter storage DB 124 indicates a weight (degree of influence) on the psychological state obtained to be the estimation result about the mood of the user B in a time interval related to an associated segment ID. Thus, by referring to each a stored in the estimation parameter storage DB 124 in step S830, the user B or the like can know which time interval the mood has a relatively large influence on the psychological state in the period T2.

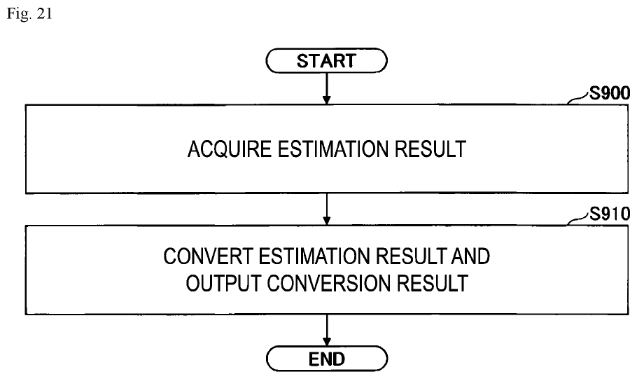

Next, the details of step S240 in FIG. 19 will be described. FIG. 21 is a flowchart illustrating an example of a processing procedure which is executed by the psychological state data restoration unit 18.

In step S900, the psychological state data restoration unit 18 acquires the estimation result (the estimation value of the psychological state) output from the psychological state estimation unit 17.

Next, the psychological state data restoration unit 18 converts the estimation result in accordance with the following conditions and outputs the conversion results (S910).

Condition 1: In a case where the value of the index to be estimated is a scalar value, a maximum value max and a minimum value min calculated by the psychological state data preprocessing unit 14 with respect to the index to be estimated and an estimation result x′ are substituted into the following equation and are converted into x having the same scale as the psychological state data recorded in the psychological state data DB 122.

x=x′(max−min)+min

Condition 2: In a case where the value of the index to be estimated is a vector value, the type of character string calculated by the psychological state data preprocessing unit 14 with respect to the index to be estimated and the index of the vector are associated with each other, and the estimation result is converted into a character string with an index having a maximum value.

As described above, the psychological state of a user, which has not been able to be estimated in the past, can be estimated, without statistically processing the mood data, by calculating the transition probability and average transition time from continuous mood data, using these pieces of data to perform learning on a model, and using the obtained model for psychological state estimation.

In addition, the psychological state of a user can be estimated with a high degree of accuracy by using effective mood transition data and mood transition time in order to estimate the psychological state of the user.

In addition, the psychological state can be estimated for each time interval corresponding to the segment ID.

From the above, according to the present embodiment, the estimation accuracy of the psychological state of a person can be improved.

Meanwhile, in the related art, because the mood has been converted into a statistic score and processed, it has not been possible to evaluate which date and time the mood has a strong influence on the psychological state. For example, it has been difficult to determine, from the statistics, whether the mood of the latest date and time from the date and time of answer to a questionnaire in order to evaluate the psychological state had a strong influence or whether the mood over the entire period had a gradual influence.

On the other hand, according to the present embodiment, the degree of importance of series data from the current to the past which is effective in estimating the psychological state of a user is automatically estimated by a self-attention mechanism, and thus the psychological state of the user can be estimated with a high degree of accuracy.

In addition, it is possible to estimate a different degree of importance with respect to the mood data depending on a time interval by using the self-attention mechanism to estimate the psychological state of the user and to understand which date and time the mood has a strong influence on the psychological state of the user by analyzing the estimated degree of importance.

Meanwhile, in the present embodiment, the mood transition probability calculation unit 12 is an example of a first calculation unit and a third calculation unit. The mood transition time calculation unit 13 is an example of a second calculation unit and a fourth calculation unit. The psychological state estimation model learning unit 16 is an example of a learning unit. The psychological state estimation unit 17 is an example of an estimation unit. The period T1 is an example of a first period. The user A is an example of a first person. The period T2 is an example of a second period. The user B is an example of a second person. The mood data series acquired in step S300 in the learning phase is an example of a first time-series data. The mood data series acquired in step S300 in the estimation phase is an example of a second time-series data.

Hereinbefore, although the embodiment of the present disclosure has been described, the present disclosure is not limited to such a specific embodiment and can be modified and changed variously without departing from the scope of the present disclosure described in the appended aspects.

### REFERENCE SIGNS LIST

10 Psychological state analysis apparatus
11 Mood data preprocessing unit
12 Mood transition probability calculation unit
13 Mood transition time calculation unit
14 Psychological state data preprocessing unit
15 Psychological state estimation model construction unit
16 Psychological state estimation model learning unit
17 Psychological state estimation unit
18 Psychological state data restoration unit
100 Drive device
101 Recording medium
102 Auxiliary storage device
103 Memory device
104 Processor
105 Interface device
121 Mood data DB
122 Psychological state data DB
123 Psychological state estimation model DB
124 Estimation parameter storage DB
B Bus

1. A psychological state analysis method comprising:
calculating, by a computer, probabilities of a plurality of mood transitions based on first time-series data on a mood of a first person in a first period;
calculating, by the computer, average persistence times of the plurality of mood transitions based on the first time-series data; and
performing, by the computer, learning on a neural network that estimates a psychological state of a person based on a vector including the probabilities of the plurality of mood transitions and a vector including the average persistence times of the plurality of mood transitions, based on a probability of the probabilities calculated for each of the plurality of mood transitions, an average persistence time of the average persistence times calculated for each of the plurality of mood transitions, and data indicating, per time interval, the psychological state of the first person in the time interval that is obtained by dividing the first period into a plurality of parts.

2. The psychological state analysis method according to claim 1, further comprising:
calculating, by the computer, probabilities of a plurality of mood transitions based on second time-series data on a mood of a second person in a second period;
calculating, by the computer, average persistence times of the plurality of mood transitions based on the second time-series data; and
estimating, by the computer, a psychological state of the second person in the second period by inputting a probability of the probabilities calculated for each of the plurality of mood transitions based on the second time-series data an average persistence time of the average persistence times calculated for each of the plurality of mood transitions based on the second time-series data to the learned neural network.

3. The psychological state analysis method according to claim 2, wherein, in estimating the psychological state of the second person in the second period, a self-attention mechanism included in the neural network is used to calculate a degree of importance per time interval that is obtained by dividing the second period into a plurality of parts and output the degree of importance.

4. A psychological state analysis apparatus comprising:
a processor; and
a memory storing program instructions that cause the processor to:
calculate probabilities of a plurality of mood transitions based on first time-series data on a mood of a first person in a first period;
calculate average persistence times of the plurality of mood transitions based on the first time-series data; and
perform learning on a neural network that estimates a psychological state of a person based on a vector including the probabilities of the plurality of mood transitions and a vector including the average persistence times of the plurality of mood transitions, based on a probability of the probabilities calculated for each of the plurality of mood transitions by the first calculation unit, an average persistence time of the average persistence times calculated for each of the plurality of mood transitions, and data indicating, per time interval, the psychological state of the first person in the time interval that is obtained by dividing the first period into a plurality of parts.

5. The psychological state analysis apparatus according to claim 4, wherein the memory further cause the processor to:
calculate probabilities of a plurality of mood transitions based on second time-series data on a mood of a second person in a second period;
calculate average persistence times of the plurality of mood transitions based on the second time-series data; and
estimate a psychological state of the second person in the second period by inputting a probability of the probabilities calculated for each of the plurality of mood transitions based on the second time-series data and an average persistence time of the average persistence times calculated for each of the plurality of mood transitions based on the second time-series data to the learned neural network.

6. The psychological state analysis apparatus according to claim 5, wherein the memory cause the processor to estimate the psychological state of the second person in the second period using a self-attention mechanism included in the neural network to calculate a degree of importance per time interval that is obtained by dividing the second period into a plurality of parts and output the degree of importance.

7. A non-transitory computer-readable storage medium that stores therein a program causing a computer to execute the psychological state analysis method of claim 1.
